(19) United States
(12) Patent Application Publication
(10) Pub. No.: US 2007/0053970 A1
(43) Pub. Date: Mar. 8, 2007

(54) LIPOSOMAL FORMULATION FOR ORAL ADMINISTRATION OF GLUTATHIONE (REDUCED) AND/OR METHYLCOBALAMINE FOR DISEASES RELATED TO GLUTATHIONE DEFICIENCY AND DEFICIENCY OF THE METHIONINE REMETHYLATION PATHWAY

(76) Inventor: F. Timothy Guilford, Palo Alto, CA (US)

Correspondence Address: BROOKE SCHUMM III, ONE NORTH CHARLES STREET, SUITE 2450, BALTIMORE, MD, 21201, US

(21) Appl. No.: 11/420,168

(22) Filed: May 24, 2006

Related U.S. Application Data
(60) Provisional application No. 60/594,996, filed on May 25, 2005.
(60) Provisional application No. 60/803,074, filed on May 24, 2006.

(51) Int. Cl. A61K 38/05; A61K 9/127; A61K 31/714; A61K 38/30

(52) U.S. Cl. 424/450; 514/18; 514/12; 514/52

(57) ABSTRACT

The invention proposes the use of reduced glutathione encapsulated in a liposome (liposomal reduced glutathione) for the oral administration of a therapeutically effective amount to improve symptoms in disease states related to diminished function of the methylation pathway such as autism, other neurodegenerative diseases and abnormalities of neurotransmitter levels in urine or blood. The invention also proposes combining liposomal encapsulated methylcobalamine, and/or liposomal encapsulated Insulin Growth Factor 1 (IGF-1) with liposomal reduced glutathione to accomplish such improvement.

### CONTINUATION DATA

This application is a continuation-in-part of U.S. Provisional Application 60/594,996 filed on May 25, 2005 with the title “The Use Of A Liposomal Formulation For Oral Administration Of Glutathione (Reduced) And/Or Methylcobalamine In The Treatment Of Diseases Related To Glutathione Deficiency And Deficiency Of The Methionine Remethylation Pathway” and 60/803,074 filed on May 24, 2006, having the same name as this application.

### SUMMARY OF INVENTION

The invention relates to a composition and method for administering liposomal reduced glutathione and methylcobalamine, and/or IGF-1 to restore methylation pathway function associated with various diseases.

The invention proposes the use of reduced glutathione encapsulated in a liposome (liposomal reduced glutathione) for the oral administration of a therapeutically effective amount to improve symptoms in disease states related to diminished function of the methylation pathway, and also proposes combining methylcobalamine, and/or Insulin Growth Factor 1 (IGF-1) with liposomal reduced glutathione to accomplish such improvement.

### TECHNICAL FIELD

The invention relates to the field of delivery of one or more nutrient substances, whose deficiency is known to cause a decrease in the function of the biochemical pathway involved in the remethylation of methionine. The delivery of the nutrients in a liposome for oral consumption facilitates their usage, uptake and utilization. The nutrients, in combination with reduced glutathione in the biochemically-reduced form, include methylcobalamine and/or Insulin Growth Factor 1. The nutrients may be administered individually or together in a liposomal preparation that allows oral delivery of a sufficient amount to improve the condition of a disease state related to deficient function of the pathway related to the remethylation of methionine. The delivery of the liposome complex of the invention may also be accomplished via absorption across the mucosa of the nose, mouth, gastrointestinal tract, after topical application for transdermal, or intravenous infusion of with or without liposome encapsulation.

The biochemical pathway involved with the metabolism of methionine to form S-Adenosylmethionine, homocysteine and the continuation of the cycle to form other biochemicals plays a central role in the metabolism of all mammalian cells. The methylation pathway depicted in FIG. 1 refers to the movement of a methyl group, a single carbon atom with its attendant hydrogens, from one amino acid to form another and is often referred to as the methionine cycle. FIG. 1.

The first compound formed in the cycle is S-Adenosylmethionine, also known as SAM or SAMe. SAMe acts as a precursor molecule to 3 main pathways: methylation, transulfuration, and aminopropylation. Two of the pathways are also coordinated by S-adenosylmethionine, which acts as an allosteric inhibitor of the methylenetetrahydrofolate reductase reaction and as an activator of cystathionine beta-synthase (Selhub). Because of this central role, SAMe ultimately plays a role in numerous methylation reactions catalyzed by methyltransferase enzymes including the synthesis of hormones, neurotransmitters, nucleic acids, proteins, and phospholipids (Mischoulon).

Coenzymes are cofactors upon which comparatively large and complex enzymes absolutely depend for their function. Coenzyme Q10 also known as ubiquinone, is the coenzyme for mitochondrial enzymes I, II and III. These mitochondrial enzymes used in the oxidative phosphorylation pathway associated with aerobic metabolism, are essential for the production of the high-energy phosphate, adenosine triphosphate (ATP), upon which all cellular functions depend. While ubiquinone is a co-enzyme required for the function of oxidative phosphorylation, it is not considered a vitamin as it is synthesized from other amino acids. However, the formation of ubiquinone requires methyl donation from SAMe (Meganathan).

SAMe also serves as a precursor molecule to the aminopropylation pathway, which leads to the synthesis of polyamines, and the transulfuration pathway, which leads to the synthesis of glutathione. (Bottiglieri).

Alterations in the function of the methionine cycle can lead to disease states. Inadequate availability of SAMe can lead to loss of methylation of phospholipids, proteins, DNA, RNA, and other small molecules (Chiang).

The identification of the elevation of the biochemical homocysteine as an independent risk factor for cardiovascular disease in men and women has focused additional attention on the methionine cycle (Aguilar). Homocysteine (Hcy) is a non-protein forming amino acid (aa) derived from the loss of the methyl group found within methionine. Normally this material is used in one of two pathways. The first is a pathway called sulfuration, where through cystathionine-beta-synthetase (CBS) it irreversibly forms cystathionine, a precursor of cysteine and glutathione. The second pathway for homocysteine is to undergo remethylation to form methionine (FIG. 1).

The methionine remethylation step can be facilitated by several intermediates such as the well known reaction mediated by folic acid and its active metabolites to form 5-methyltetrahydrofolate (5-MTHF) creating tetrahydrofolate (THF) which will then regenerate to 5-MTHF through the action of methylenetetrahydrofolate reductase (MTHFR).

Elevations of homocysteine can occur from inhibition of the remethylation route or inhibition or saturation of the transsulfuration pathway. The main factors that are generally associated with elevations of homocysteine are nutritional deficiency, particularly related to folate metabolism, mutations of the enzymes MTHFR and CBS (Aguilar).

While attention has been focused on the elevation of homocysteine recent research points out that the methionine cycle can be compromised at other points leading to deficiencies with normal or low levels of homocysteine. Elevations of the intermediate S-Adenosylhomocysteine (SAH) lead to a feed back inhibition of the function of the cycle and also a reduction in the transsulfuration pathway leading to a deficiency in the production of cysteine and glutathione (James). Deficiency of glutathione alone will lead to many disease presentations as glutathione is involved in many cell functions as an antioxidant (Lieber), (Warner), a detoxifying agent (Cersosimo) and a cell signal (Peterson), (Droge), (Dickinson). Glutathione serves as a platform for the antioxidant enzyme glutathione peroxidase, which functions to convert peroxide, H2O2 to water, H2O. This reaction functions in conjunction with the mineral selenium. In order to improve the function of the enzyme glutathione peroxidase, adequate selenium in the mammalian system is important, and may be needed as a supplement.

The tripeptide L-glutathione (GSH) (gamma-glutamyl-cysteinyl-glycine) is well known in biological and medical studies to serve several essential functions in the cells of higher organisms such as mammals. It is functional when it appears in the biochemical form known as the reduced state (GSH). When oxidized, it forms into a form known as a dimer (GSSG), and after interaction with a reducing agent such as vitamin C, can returned to the active state, without loss of function.

Aerobic energy metabolism leads to the production of reactive oxygen species (ROS). The levels of ROS maintained by an oxidant-antioxidant balance within each cell serves as important signaling molecules influencing cellular activities such as T cell activation, vasomotor tone, and normal gene expression (Kamata).

When cellular antioxidant defense mechanisms fail to counterbalance and control ROS production damage to surrounding molecules causing peroxidation of lipid membranes and proteins. Accumulation of these materials and other toxins leads to cell damage and eventually to cell death and compromise of tissue function. This situation is known as oxidation stress. The lack of sufficient glutathione in the reduced state relative to the oxidized state may be due to lack of production of glutathione (reduced) or an excess of the materials such as toxins that consume glutathione (reduced). The lack of glutathione (reduced) may manifest as a systemic deficiency or locally in specific cells undergoing oxidation stress.

Deficiency of glutathione in the reduced state contributes to oxidative stress, which has been documented to play a key role in aging and the pathogenesis of many diseases such as
Autism
Cystic fibrosis
Liver disease
Parkinson's disease
Alzheimer's disease
Heart attack and Stroke
Diabetes
Viral disease

Glutathione is formed from three amino acids, glutamine, which is found in food protein sources such as meats, glycine, which is found in high enough concentrations in all foods and cysteine, which is the rate limiting factor in the formation of glutathione. Cysteine is not considered an essential amino acid. An essential amino acid is one that must be provided from outside sources, and because cysteine can be formed from methionine, it is not cysteine is not considered essential. Methionine is found readily in foods.

The biochemical pathway related to the recycling of methionine can be interrupted by a number of situations including states of excess oxidation (oxidation stress), toxic metals such as mercury and ethanol exposure (Waly). These biochemical stressors are generally removed from the body by glutathione for remediation. In what seems like a twist of biochemical irony, glutathione, which is a product of the methionine/transsulfuration cycle is actually needed to maintain the function of the methionine cycle. Thus, toxins or blockades that lead to decreased glutathione function will lead to an ever spiraling decrease in the formation of glutathione. In this situation an outside or exogenous source of reduced glutathione may be needed for some time to resuscitate the compromised methionine cycle and allow the normal formation of glutathione. It is the purpose of the invention to use liposomal encapsulation of glutathione individually or in combination with methylcobalamine and/or Insulin Growth Factor 1 (IGF1) to reconstitute or salvage the decreased function of the methylation of methionine pathway caused by mercury, other toxins and oxidation stress.

The preferred embodiment of the combined administration of reduced glutathione and IGF1, as to the IGF1, is a liposome encapsulation of the IGF1 in a spray delivery system. The IGF1 in the preferred embodiment is from a natural source of IGF1 such as an extract from deer antler velvet (Cervi parvum Cornu). The spray is designed to release 11 mg of the extract for each serving size of 2 sprays. This yields approximately 27.8 nanograms (ng) per serving (a total of 2500 nanograms (ng) of natural IGF1 is contained in each bottle). Preferred administration is 2 sprays 3 times a day. Cervi parvum Cornu is available in a natural source available in a spray delivery system in liposome application from Biozone, Inc., 580 Garcia Ave, Pittsburg, Calif. (USA) 94565.

The invention also includes the use of recombinant IGF1 for the IGF1 component. Recombinant IGF1 is available from Sigma-Aldridge company St. Louis, Mo. The term IGF1 includes the liposome encapsulation of IGF1, and recombinant IGF1 in liposomal form. The term IGF1 includes the recombinant form of IGF1.

The blockade of the formation of glutathione from the remethylation of methionine cycle can lead to a variety of disease states. Abnormality in this cycle has been demonstrated to be related to deficiency of methylated B12 (James). Glutathione has been shown to protect B12 from degradation by pollutants, increasing its availability for the continuation of the methylation reaction (Watson). Glutathione is required for the methylation of B12, and thus the continued formation of related products (Xia). Deficiency of glutathione thus results in deficiency of methylcobalamine and a number of disease states such as
Autism
Neurodevelopmental disability
   Attention Deficit Disorder
   Attention deficit, Hyperactivity Disorder
Neurodegenerative disease such as neuropathy and Alzheimer's disease.
Neurogenerative disease such as Amyotrophic lateral sclerosis
Neurological disease such as neuropathy.
Decreased glutathione in the brain of individuals with encephalitis or multiple sclerosis with normal or increased blood levels of glutathione
Neurotransmitter deficiency
Clinical Effects of neurotransmitter deficiency such as depression, anxiety, malaise and low energy.
Homocysteine accumulation in serum
Decreased synthesis of cysteine (James)

The interruption of the formation of glutathione by the described series of events at the present time has no single term describe the deleterious events that occur after inhibition of the methionine cycle. The decrease of the availability of glutathione that accompanies mercury exposure becomes compounded by the fact that mercury is removed by glutathione in the normal situation. As the events continue to unfold any other toxic or oxidative stress experience with an impact on these biochemical reactions conspire to further reduce the availability of glutathione. As glutathione is needed for the methylation of methionine cycle to function efficiently, a number of health problems may develop. The individual affected can be caught in a continuing downward spiral of declining health. The invention is designed to restore or salvage the pathway to more normal function and return the individual to an improved state of health.

A term coined and claimed in the invention to describe this situation of declining health related to the conspiring events leading to decreased function of the methylation cycle is conspiral. It is used to describe these events in such a usage as the mercury conspiral. The con refers doubly to both the conspiring causative events, and also connotes the negative impact of the events creating the downward spiral of health. The events leading to dysfunction of the methionine cycle due to lack of glutathione as well as direct effects on methionine synthase conspire to create a state of being that is contrary to good health.

Components of the remethylation reactions require various vitamins to complete the reaction. These vitamins have been documented to include folic acid, and its active metabolite, folinic acid, as well as cyanocobalamin (vitamin B-12) and its active metabolite methylcobalamine. Betaine can be of additional benefit in this pathway as a source of methyl groups for the methylation reaction.

Autism is a neurodevelopmental disability that is usually diagnosed in the early years of life, often before age 3. Autism is characterized by a number of difficulties including deficits in social communication and language skills. There are associated repetitive behaviors and restriction of interests. In addition to the behavioral impairment autism is associated with a high incidence of gastrointestinal disease and dysbiosis, autoimmune disease and mental retardation.

Deficiency of glutathione in the reduced state, or an excess of oxidized glutathione has been demonstrated to be present in children with autism in a study by James (James). There were also differences between controls and children with autism in the intermediate metabolites of the remethylation of methionine cycle.

TABLE 1
Comparison of methionine cycle and transsulfuration metabolites between
autistic children and control children

| | Control children | Autistic children |
|---|---|---|
| | (n_33) | (n_20) |
| Methionine (μmol/L) | 31.5 ± 5.7 (23-48) | 19.3 ± 9.7 (15-25) |
| SAM (nmol/L) | 96.9 ± 12 (77-127) | 75.8 ± 16.2 (68-100) |
| SAH (nmol/L) | 19.4 ± 3.4 (16-27) | 28.9 ± 7.2 (14-41) |
| SAM:SAH | 5.2 ± 1.3 (4-8) | 2.9 ± 0.8 (2-4) |
| Adenosine (μmol/L) | 0.27 ± 0.1 (0.1-0 | 0.39 ± 0.2 (0.17-0.83) |
| Homocysteine (μmol/L) | 6.4 ± 1.3 (4.3-9.0) | 5.8 ± 1.0 (4.0-5.8) |
| Cystathionine (μmol/L) | 0.17 ± 0.05 (0.1-0.27) | 0.14 ± 0.06 (0.04-0.2) |
| Cysteine (μmol/L) | 202 ± 17 (172-252) | 163 ± 15 (133-189) |
| tGSH (μmol/L) | 7.6 ± 1.4 (3.8-9.2) | 4.1 ± 0.5 (3.3-5.2) |
| Oxidized glutathione | 0.32 ± 0.1 (0.11-0.43) | 0.55_0.2 (0.29-0.97) |
| (nmol/L) | | |
| tGSH:GSSG | 25.5_8.9 (13-49) | 8.6_3.5 (4-11) |

Supplementation of autistic children with twice daily administration of 800 μg folinic acid and 1000 mg betaine over a 3 month period was effective in normalizing methionine cycle metabolites to the concentrations in controls. The intervention with folinic acid and betaine improved but did not normalize total GSH (tGSH) or GSSG concentrations or the tGSH:GSSG ratio. A second intervention that added injectable methylcobalamine resulted in a further decrease in the concentration of adenosine and GSSG and a further increase in the concentrations of methionine, cysteine, and tGSH and SAM:SAH and tGSH:GSSG ratios. While the James study has shown improvement, practical experience and clinical observation has shown that many children using methylcobalamine develop substantially increased irritability after using methylcobalamine by injection. There is preliminary data suggesting that methylcobalamine by itself can cause an increase in SAH, leading to incomplete reconstitution of the methionine cycle.

### DESCRIPTION OF FIGURES

FIG. 1 shows Remethylation of Methionine and Transulferation Pathways.

FIG. 2 shows the requirement for glutathione to allow the formation of methylcobalamine

FIG. 3 shows that the methylation of methionine is dependent on glutathione for normal function. FIG. 3 is used to illustrate the mechanism of the interaction between glutathionyl cobalamin and 5-methyltetrahydrofolate to form methylcobalamine.

FIG. 4 shows that of the three amino acids involved in glutathione production, glutamate, cysteine, and glycine, cysteine is usually considered to be the limiting resource.

FIG. 5 shows that glutathione is produced in a separate reaction from the methionine cycle.

### DESCRIPTION OF INVENTION

The use of liposomal glutathione as described in the present invention alleviates this condition. The preferred dosing liposomal glutathione to avoid this situation for children is 100 mg. for every 30 pounds of weight placed in 4 to 8 ounces of liquid such as water or any beverage of choice and ingested immediately or over a 4 to 6 hour period. See dosing example for adult dose, and case example 3. The continuous presence of glutathione throughout the day allows for diminishing the level of toxin or oxidation stress that has compromised the function of the methionine pathway. Thus glutathione alone or in combination with methylcobalamine and, in come cases, IGF1 is the preferred embodiment of the invention.

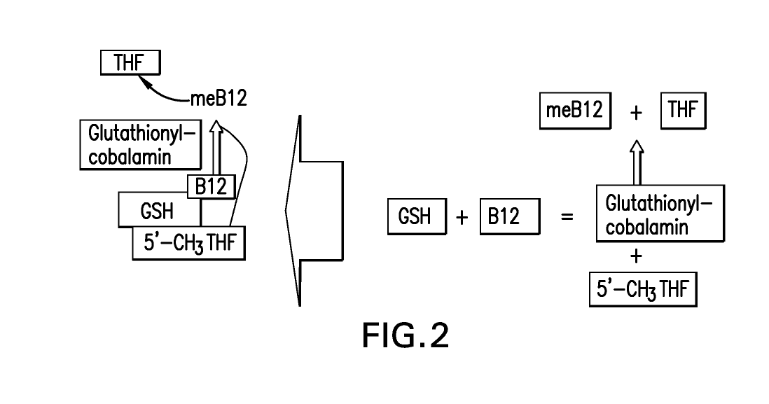

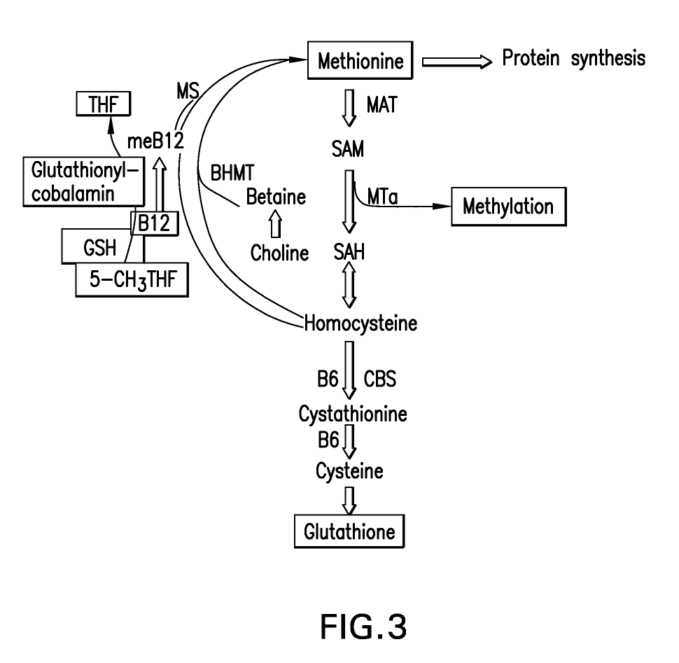

While not readily apparent, the requirement for glutathione to allow the formation of methylcobalamine See FIG. 2, means that the methylation of methionine is dependent on glutathione for normal function. See FIG. 3.

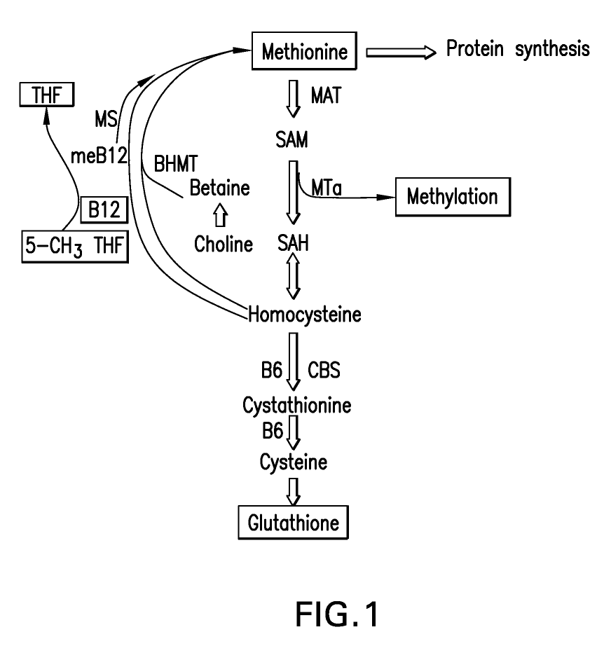

Prior to the implementation of the present invention the pathway for methylation of methionine was thought to go forward as described in FIG. 1. After implementation of the invention it has become clear that the pathway should be described as seen in FIG. 2, that is, requiring glutathione for the cycle to function. FIG. 3 is used to illustrate the mechanism of the interaction between glutathionyl cobalamin and 5-methyltetrahydrofolate to form methylcobalamine.

This invention claims the use of reduced glutathione, with the preferred embodiment the liposomal encapsulation described, for use in disease situations that have occurred along with or secondary to inefficient function of the methionine methylation pathway to enable the restoration of the methionine cycle in disease states such as, but not limited to, autism. The intention is to restore or salvage the abnormalities that have occurred due to the biochemical deficiencies in the pathway by the administration of glutathione, which is key to the restoration of the function of the pathway.

The need for the cycle between methionine and homocysteine to function in a repetitive fashion is illustrated by the fact that in men the homocysteine moiety is recycled between methionine and homocysteine twice before being converted to cystathionine, but in women it is recycled about 1.5 times (Chiang). This may contribute to the observation that methionine cycle dependent syndromes such as autism occur in male with a higher frequency of 4:1 compared to females. This finding suggests that there is a greater need for providing the invention in men than women in order to maintain health.

At the same time, it points out the need for the cycle to function in a continuous fashion. Thus, this pathway must function on a continuous basis to function efficiently, and requires the constant presence of adequate glutathione as well as the formation of methylcobalamine

The use of the glutathione in the present invention is necessary to reduce the oxidation stress that accompanies illnesses that decrease the methionine pathway function. Without the addition of glutathione, the pathway, briefly reconstituted by the addition of methylcobalamine alone will sputter, like an unused engine, creating inefficient function, and an imbalance of related methylation reactions producing an abundance of pathway inhibiting substances like SAH. This observation and the need for continuous function explains why the intermittent addition of methylcobalamine in injection form does not always restore the cycle to an efficient function.

The use of the S-adenosyl methionine and its constituents both in the methionine pathway and in related pathways has been referenced by Schwartz, U.S. Pat. No. 6,596,701. The Schwartz patent references the use of the measured levels of constituents of the pathway as indicators of the need to administer the constituents of the SAM pathway, and references cysteine and glutathione as an indicator of dysfunction of the SAM pathway. However, glutathione is not part of the SAM pathway, and while produced from a product of a product of the SAM cycle, ultimately glutathione is formed outside the SAM/methionine cycle. While it is dependent on the cycle to produce the precursor to the cysteine component, glutathione itself requires a separate pathway for its production. This pathway is separate from the methionine cycle. However, there is no previous reference for the use of glutathione to reconstitute the compromised methionine pathway.

Glutathione is produced in a separate reaction from the methionine cycle, Illustrated in FIG. 5. While cysteine, which is formed by the transsulfuration pathway from homocysteine and is therefore an indirect measure of the function of the methionine cycle, glutathione formation requires additional steps for its formation and is not directly a component of the cycles related to S-adenosyl methionine.

Similarly, the Schwartz patent, U.S. Pat. No. 6,596,701, while references that replacement of deficiencies of the constituent components related to the formation of S-adenosyl methionine will aid in the restoration of health related problems, there is no references for the use of the combination of glutathione alone or in combination with methylcobalamine to restore the function of the methionine cycle.

Additional patents have referenced the use of S-adenosyl methionine in various manifestations for the treatment of disease, however none of these references the use of either glutathione or methylcobalamine for the purpose of restoring the methionine cycle. Nor do they reference the use of glutathione to “prime the pump” of the methionine cycle to produce the cysteine that is needed to form glutathione, as the present invention claims.

A patent, Smith, U.S. Pat. No. 6,764,693, Jul. 20, 2004, references the use of liposomes containing a combination of glutathione with at least one other antioxidant material to increase intracellular and extra cellular antioxidants. The Smith patent references the treatment of disease by restoration of antioxidant function, but does not reference improvement by restoring the function of the remethylation of methionine, not does it reference the use of reduced glutathione as a single entity in a liposome. Neither methylcobalamine nor IGF-1 is in the class of compounds referred to as antioxidants.

Demopoulos, U.S. Pat. No. 6,350,467, references the use of glutathione and ascorbic acid to influence the redox status of cells in disease states, however does not reference the use of a liposome encapsulation to deliver and maintain the glutathione in the reduced state to the system. The Demopoulos patent also references as the preferred embodiment of the invention the combination of glutathione and ascorbic acid which is needed to maintain the reduced state of the glutathione and to facilitate its function. The present invention claims the use of reduced glutathione in a liposome encapsulation to facilitate absorption as well as to maintain the glutathione in the reduced state.

The pattern observed in children with autism demonstrating decreased concentrations of cysteine, cystathionine and tGSH are consistent with reduced efficiency of the transsulfuration pathway. The increase in GSSG disulfide and the decrease in the ratio of tGSH:GSSH found indicate chronic oxidation stress.

Within the methionine cycle several of the enzymes are vulnerable to oxidative stress. These include methionine synthase, betaine homocysteine transferase, and methionine adenosyltansferase, which are redox-sensitive enzymes and are down-regulated by oxidative stress. A decrease in the activity of these enzymes would result in a decrease in the formation of cysteine, effectively making it an essential amino acid in individuals with impairment of these pathways.

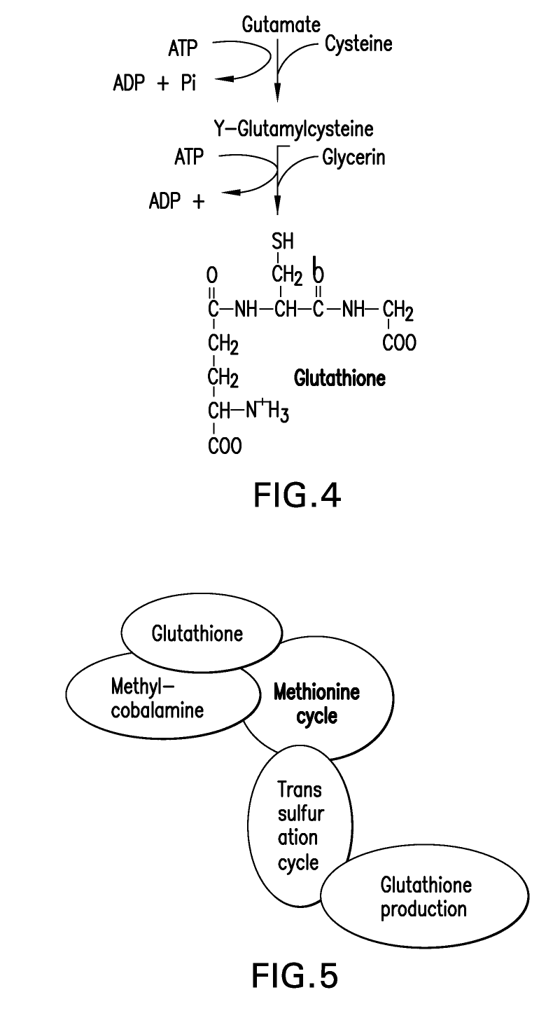

Glutathione synthesis is controlled by the first enzyme in the synthetic pathway, glutamate-cysteine ligase (GCL), otherwise known as -glutamylcysteine synthetase, which creates -L-glutamyl-L-cysteine (GC). Of the three amino acids involved in glutathione production, glutamate, cysteine, and glycine, cysteine is usually considered to be the limiting resource (Meister). FIG. 4.

Because cysteine is the rate-limiting amino acid for glutathione synthesis, a decrease of cysteine will result in the low glutathione levels seen in individuals with impairment of this pathway. Impairment of the formation of glutathione results in increased vulnerability to oxidative stress.

Methylcobalamine given by injection therapy in children with autism has been observed to result in improvement in speech and cognition in children with autism (James). After a month of injection therapy children with autism were not only demonstrating improvement in communication, but the level of total GSH (tGSH) was increased and GSSG decreased. This resulted in normalization of the tGSH:GSSG ratio, also known as the glutathione redox profile. It is thought the improvement resulted from the increase in the availability of cysteine, as the rate-limiting precursor for glutathione synthesis (James).

During chronic oxidation stress there is a higher demand for the production of glutathione and thus, there is an increased demand placed on the methionine and transsulfuration pathways. The vulnerability of these pathways to oxidation stress can lead to a decline in function in affected individuals. While these pathway inefficiencies have been demonstrated in autism, it is likely that similar defects will affect older individuals and may present with a different set of symptoms. The sources of the oxidation stress could be environmental, intracellular or both.

Restoring the cycle of remethylation of methionine appears to be of significant benefit in disease states associated with disruption of this cycle. Autism represents such a disease situation, and many others may exist. While many of the materials used to restore the methionine cycle are biologically available by oral absorption, methylcobalamine and glutathione are not absorbed in a pure or “neat” oral form. The purpose of the present invention is to allow the administration of methylcobalamine in a liposomal encapsulation that facilitates absorption after oral ingestion. A liposomal encapsulation of reduced glutathione has already been described in Guilford in a provisional patent application Ser. No. 60/522,785 on Nov. 7, 2004 entitled “Liposomal Formulation for Oral Administration of Glutathione (Reduced)”. The present invention also describes the use of liposomal methylcobalamine and/or glutathione in disease states characterized by compromise of the methionine cycle and transsulfuration pathway.

The use of the term “glutathione” or “glutathione (reduced)” will refer to glutathione in the reduced state.

The inventor Guilford filed a provisional application Ser. No. 60/522,785 on Nov. 7, 2004 entitled “Liposomal Formulation for Oral Administration of Glutathione (Reduced)” which is adopted and incorporated herein by reference. As reviewed in the previous application, it has been demonstrated in a clinical study that 3 grams of glutathione delivered by oral ingestion does not elevate plasma glutathione levels (Witschi).

A liposome is a microscopic fluid filled pouch whose walls are made of one or more layers of phospholipid materials identical to the phospholipid that makes up cell membranes. Lipids can be used to deliver materials such as drugs to the body because of the enhanced absorption of the liposome. The outer wall of the liposome is fat soluble, while the inside is water-soluble. This combination allows the liposome to become an excellent method for delivery of water-soluble materials that would otherwise not be absorbed into the body.

Administration of methylcobalamine can be accomplished by injection or the use of sublingual tablets that dissolve slowly on the mucosa under the tongue. This area is noted for its ability to absorb materials. There are however several problems associated with the sublingual administration route including

Lack of Assurance of Uniform Absorption

1. A significant segment of the population is either unable or unwilling to allow the tablet to remain in place long enough to allow dissolution and absorption to occur. This is particularly true in the pediatric population, but also occurs in adults, especially older adults.

2. While tableted or other solid forms of administration of nutrients is convenient for many individuals there is a significant segment of the population for whom swallowing a tablet is not possible. This can be due to age, such as the pediatric segment of the population or the other end of the age spectrum, the geriatric population, many of whom find pill swallowing difficult.

Injection of methylcobalamine results in uniform administration, but is not an acceptable route of administration for many individuals due to aversion to repeated needle sticks. As the material may need to be administered more than once a day, and for a prolonged period of time, the use of injections using needles becomes even less attractive.

For this reason, as well as ease of dose calculation, liquid gel delivery of glutathione or methylcobalamine will be more universally acceptable. Another advantage is that the present invention enables administration of a larger quantity of either GSH of methylcobalamine in a single dose than other forms of non-parenteral administration as well as enabling incremental adjustment of doses for children and adults.

The administration of the methylcobalamine will be particularly easy and effective to administer as the dosing is such that it can be administered in a spray. For example a 500 microgram (mcg.) dose can be administered in a single spray of 0.65 mg (which because it is approximately equal to 0.65 ml is better thought of in milliliters) in the preferred embodiment of the invention.

For doses smaller than 500 mcg, the spray cap can be removed and a cap adapter that fits an oral syringe can be used. The oral syringe can be of a size from 1 to 3 ml to allow very accurate measurement of quantities less than 500 mcg. For example for a 250 mcg dose, the syringe could be used to measure 0.325 ml of the preferred concentration of liposomal methylcobalamine.

Liposome delivery of glutathione as described in this invention is particularly efficient for providing glutathione to the immune system. The macrophage cells have been demonstrated to have a preference for adsorption of liposomes. Increased adsorption improves the delivery of glutathione to macrophages which are involved in the immune decision regulating two sides of the immune response known as TH1 and TH2. TH1 represents the immune response utilizing cell mediated immunity. TH2 represents the use of antibody formation and the response known as chronic inflammation. Ideally, the two responses work together to alert the system of an invader with the attachment of antibodies to an invader (TH2) and signal the removal of the antibody-invader complex by an engulfing and killing T cell activity (TH1). In the presence of excess oxidation characterized by a deficiency of glutathione the response is typified by an excess of the reaction known as TH2. The response is also known as chronic inflammation. The reaction products associated with chronic inflammation can cause an excess of damage to the normal tissues while the invader is contained.

Examples of chronic inflammatory diseases include allergy, asthma, autoimmune disease, and vascular disease. Autism is associated with allergies, autoimmune disease and gastrointestinal inflammatory disease (James). Administration of liposomal glutathione has been demonstrated to improve these symptoms in children with autism (See examples).

The invention is claimed for the improvement of the methionine cycle in a variety of disease states in addition to autism. These include Neurotransmitter deficiency states. For purposes of this invention, neurotransmitter deficiency states include: mood disorders characterized by deficiencies in one or more of serotonin, dopamine, norepinephrine, and epinephrine. Further, a neurotransmitter deficiency state including the elevation of GABA (gamma-amino-benzoic acid), PEA (phenyl-ethyl-amine), and histamine, and more generally any abnormal alteration of excitatory or inhibitory neurotransmitters that result in a disease state. Generally speaking, there are two main types of neurotransmitters: Excitatory and Inhibitory.

Excitatory neurotransmitters act on receptors which increase the neurons ability to respond and relay incoming messages. Inhibitory neurotransmitters reduce neuronal excitability and increase the likelihood that an incoming signal will be terminated. Excessive input from the excitatory system can lead to insomnia, anxiety, irritability and even seizures. Excess of the inhibitory neurotransmitters can lead to sedation, dullness, incoordination and even anesthesia.

Dopamine, norepinephrine, epinephrine are considered excitatory neurotransmitters, while serotonin and GABA are considered inhibitory.

The assessment portion of the invention includes measurement of Neurotransmitter status using laboratory measurement of the excretion of neurotransmitters in the urine. The preferred method of assessment includes

a. Dopamine

b. Norepinephrine

c. Epinephrine

d. Serotonin

e. Gamma-Amino Benzoic acid (GABA)

f. Glutamate

g. Phenylethylamine (PEA)

h. Histamine

SAMe is required for the formation of dopamine, norepinephrine, epinephrine and serotonin (Mischoulon). Methylation is required for the formation of epinephrine from norepinephrine and is also involved in the catabolism of histamine. Observations on the formation of the various materials represent a method of determining the efficiency of the methionine cycle and the need utilize the present invention. See Example Case 3

In case 3, an individual with documented vitamin B12 deficiency the neurotransmitter findings were:

Epinephrine 4.2 μg/go cretonne (8-12)

Norepinephrine 26.6 μg/grocer (30-55)

Dopamine 89.2 μg/grocer (125-175)

Serotonin 48.6 μg/grocer (175-225)

GABA 7.0 μmol/grocer (2.0-4.0)

Glutamate 45.3 μmol/grocer (10-25)

PEA 529.70 nmol/grocer (175-350)

Histamine 82.7 μg/grocer (10-22)

Cretonne 35.0 mg/do

Glutathione in RBC 38 μmol/L (200-400)

The results show that the individual is low in epinephrine, norepinephrine, dopamine and serotonin, all related to diminished methionine cycle function and decreased methylation. The elevation of histamine is also an indicator of decreased methylation function as it is inactivated by histamine-methyl transferase.

The individual did not improve using the commonly known intermediates and support supplements of the methionine cycle such as B12 (cyanocobalamine) by injection, folic acid, betaine, SAMe, N-acetylcysteine, and even glutathione in the neat form (not in the form claimed in the present invention).

Upon the addition of the invention, in the form of liposomal encapsulated glutathione, the individual began to experience significant improvement in his symptoms. He reported that within hours of initiating the liposomal glutathione in a dose of 600 mg to 1200 mg in divided doses throughout the day, he began to feel better, with a decrease in his sense of impending doom and anxiety.

It is not obvious that the methionine cycle would need to supported by a biochemical like glutathione, whose formation is considered to be dependent on the function of the methionine cycle. The dependence is due to its independent formation from cysteine. Glutathione, however, is not a direct product of the methionine cycle, as it depends on the function of additional enzymes and the availability of additional components outside those of the methionine cycle. Schwartz, U.S. Pat. No. 6,596,70, has claimed that SAMe and components of the related cycles be measured and replaced, but there is no claim for maintaining the cycle by providing glutathione to maintain the cycle, and alleviate the symptoms of body dysfunction as claimed in this invention. In the example case 3 cited the individual did not experience benefit with replacement of the commonly associated methods of support of the methionine cycle and it was not until he began to take the liposomal encapsulation of glutathione that he began to improve.

The liposome preparations claimed in this invention allows the manufacture of a stable product, which can be used for the administration of glutathione in a form that is convenient. The liposome-glutathione preparation described is also stable from oxidation, allowing a two year, unrefrigerated shelf-life of the product, and has specific characteristics of uptake into cell membranes that improve its therapeutic qualities for certain disease states.

The liposomal glutathione in the preferred preparation has been demonstrated to contain over 95% reduced glutathione. Sampling of the liposomal glutathione done at the time of production has shown 96% reduced glutathione and a second example done at 4 months after preparation shows 97% reduced glutathione. Table 1

Previous use of liposomes encapsulating glutathione has been limited by concern that the combination would be adversely affected by the acidity and enzymes of the stomach. The preparation used in the present invention is able to deliver therapeutically active amounts of glutathione to the system in spite of these concerns. The invention describes the lipid encapsulation of the glutathione (reduced) or methylcobalamine into the lipid vesicle of liposomes and administered orally for the transmucosal absorption into the nose, mouth, throat or gastrointestinal tract providing the ability to conveniently supply therapeutically effective amounts of glutathione (reduced) or methylcobalamine. The invention may also be administered topically for dermal and transdermal administration as well as intravenously.

Claims for the liposomal encapsulation of the other forms of vitamin B12 are included. These forms include cyanocobalamin, hydroxycobalamin and adenocobalamin. The term methylcobalamine in the claims includes methylcobalamine and equivalently active racemers and sterioisomers and cyanocobalamin, hydroxycobalamin and adenocobalamin, and glutathionylcobalamin.

### DETAILED DESCRIPTION OF THE PREFERRED EMBODIMENTS

### Example 1

Liposomal glutathione Drink or Spray 2500 mg per ounce or
form suitable for encapsulation or gel

| | % w/w |
|---|---|
| Deionized Water | 74.4 |
| Glycerin | 15.00 |
| Lecithin | 1.50 |
| Potassium Sorbate | 0.10 |
| (optional spoilage | |
| retardant) | |
| Glutathione (reduced) | 8.25 |

A lipid mixture having components lecithin, and glycerin were commingled in a large volume flask and set aside for compounding.

In a separate beaker, a water mixture having water, glycerin, glutathione were mixed and heated to 50.degree. C.

The water mixture was added to the lipid mixture while vigorously mixing with a high speed, high shear homogenizing mixer at 750-1500 rpm for 30 minutes.

The homogenizer was stopped and the solution was placed on a magnetic stirring plate, covered with parafilm and mixed with a magnetic stir bar until cooled to room temperature. Normally, a spoilage retardant such as potassium sorbate or BHT would be added. The solution would be placed in appropriate dispenser for ingestion as a liquid or administration as a spray.

Analysis of the preparation under an optical light microscope with polarized light at 400× magnification confirmed presence of both multilamellar lipid vesicles (MLV) and unilamellar lipid vesicles.

The preferred embodiment includes the variations of the amount of glutathione to create less concentrated amounts of glutathione. The methods of manufacture described in Keller et al U.S. Pat. No. 5,891,465 are incorporated into this description.

### Example 2

Methylcobalamine combination designed to yield a spray with an
individual volume of 0.65 cc, yielding 500 mcg. per spray.

| Component | % w/w |
|---|---|
| Deionized Water | 83.325 |
| Glycerin | 15.00 |
| Lecithin | 1.50 |
| Potassium Sorbate | 0.10 |
| (optional spoilage | |
| retardant) | |
| methylcobalamine | 0.075 |

A lipid mixture having components lecithin, and glycerin were commingled in a large volume flask and set aside for compounding.

In a separate beaker, a water mixture having water, glycerin, methylcobalamine were mixed and heated to 50.degree. C.

The water mixture was added to the lipid mixture while vigorously mixing with a high speed, high shear homogenizing mixer at 750-1500 rpm for 30 minutes.

The homogenizer was stopped and the solution was placed on a magnetic stirring plate, covered with parafilm and mixed with a magnetic stir bar until cooled to room temperature. Normally, a spoilage retardant such as potassium sorbate or BHT would be added. The solution would be placed in appropriate dispenser for ingestion as a liquid or administration as a spray.

Analysis of the preparation under an optical light microscope with polarized light at 400× magnification confirmed presence of both multilamellar lipid vesicles (MLV) and unilamellar lipid vesicles.

The preferred embodiment includes the variations of the amount of glutathione to create less concentrated amounts of glutathione. The methods of manufacture described in Keller et al, U.S. Pat. No. 5,891,465 are incorporated into this description.

TABLE 1

Preparation 1
Time: 4 months after production
Oxidized glutathione: 4.177 mg/ml
Reduced Glutathione: 80.36 mg/ml
% Glutathione Reduced: 95%
Preparation 2
Time: at production
Oxidized glutathione: 2.933 mg/ml
Reduced glutathione: 84.14 mg/ml
% glutathione reduced: 96.7%

### Case Examples and Dosing

Liposomal Glutathione in Autism

Case 1.

8-Year-Old Boy

Diagnosis: Autism Spectrum Disorder.

Symptoms include: Trouble with attention; social skills that are not age-appropriate; some nervous behavior and some low-level stimming.

Background: Prior to the onset of administration of the liposomal glutathione the individual had been treated with intermittent administration of intravenous glutathione.

Duration of use of liposomal glutathione: 2 months

Questionnaire:

Did you experience any improvement in these systems?: Yes

If so, please tell us (in detail) about the improvement you've seen: Definite decrease in the nervous behavior—in fact the stimming, which was somewhat subtle, has disappeared. Better attention. More interest in playing with the children in his class. His plasma cysteine and plasma sulfate levels (measured by Great Smokies Lab) increased in the first 5 weeks from well below to almost into the bottom of the reference range. Visual Contrast Sensitivity Test score has increased.

How many teaspoons of liposomal glutathione were you taking per day when you started noticing these improvements: 1½ teaspoons (600 mg.) BID

Are you currently taking any other forms of glutathione: No

How many teaspoons are you currently taking for maintenance per day (if different from above)?: 1 teaspoon (400 mg.) BID

Do you mix liposomal glutathione with another liquid (i.e. orange juice) for ingestion: No

If so, what have you mixed it with? (Please share what liquids, and the outcomes—tasted good/bad): It tastes fine in water.

Additional Information:

Have you been on any IV glutathione regimen: Yes.

Case 2.

Condition you are using liposomal glutathione for:

1 am 79. I have neuropathy. I could not walk on hard floors or concrete. I had to use a wheelchair to shop in stores. My kitchen floor is ceramic and I couldn't even step on it without suffering with extreme pain.

Your symptoms include: My legs hurt terribly from the knees to my ankles.

How long have you been using liposomal glutathione: 12-13 weeks

User Information:

Did you experience any improvement in these systems?: Yes

If so, please tell us (in detail) about the improvement you've seen: I no longer have to use a wheelchair when shopping. I can now clean my kitchen and do the cooking. My legs still hurt if I stand too long on hard surfaces, but they are much better now.

Dose in Teaspoons of liposomal glutathione when improvements occurred: 1½ tsp (600 mg.) twice a day

Are you currently taking any other forms of glutathione: No

Have you been on any IV glutathione regimen: No

Case 3.

MM, a 46 year old man with recurring episodes of chest pain and syncope requiring emergency room evaluation on 5 occasions over the previous year. While his chest pain was relieved by nitroglycerin, administered as a coronary artery dilator, his cardiac evaluation showed no vascular abnormality. In addition he describes exhaustion, weakness, extreme agitation, anxiety, occasional vomiting, the feeling of impending doom. These symptoms were accompanied by low blood pressure of 90/60. He was unable to work for 4 months prior to undergoing the methylation pathway assessment described in the present invention.

One month previously MM was found to have a vitamin B12 level of 240 pg/ml (211-911 pg/ml). His methylmalonic acid was 366 nmol/L (88-243 nmol/L). The elevation, methylmalonic acid indicates decreased B12 function, as normal B12 function would result in a normal level of methylmalonic acid.

During the month prior to evaluation the MM initiated therapy with SAMe, folate, betaine (trimethyl glycine), N-acetylcysteine and B12 by injection and oral glutathione (neat, not in the form of the invention). A few days after initiating these therapies he actually felt worse, and developed high blood pressure with 170/120 blood pressure, and had to stop the supplements. He noted shortly after this that he developed oral thrush.

Testing done prior to initiating the present invention revealed that he had a very low RBC glutathione, and his urine neurotransmitter levels with the optimal range in parentheses:

Epinephrine 4.2 μg/go cretonne (8-12)

Norepinephrine 26.6 μg/grocer (30-55)

Dopamine 89.2 μg/grocer (125-175)

Serotonin 48.6 μg/grocer (175-225)

GABA 7.0 μmol/grocer (2.0-4.0)

Glutamate 45.3 μmol/grocer (10-25)

PEA 529.70 nmol/grocer (175-350)

Histamine 82.7 μg/grocer (10-22)

Cretonne 35.0 mg/do

The results show that the individual is low in epinephrine, norepinephrine, dopamine and serotonin, each of which is related to diminished methionine cycle function and decreased methylation. The elevation of histamine is also an indicator of decreased methylation function as it is inactivated by histamine-methyl transferase.

The individual did not improve using the commonly known intermediates and support supplements of the methionine cycle such as B12 (cyanocobalamin) by injection, folic acid, betaine, SAMe, N-acetylcysteine, and even glutathione in the neat form (not in the form claimed in the present invention).

Upon the addition of the invention, in the form of liposomal encapsulated glutathione, the individual began to experience significant improvement in his symptoms. He reported that within hours of initiating the liposomal glutathione in a dose of 1200 mg in divided doses throughout the day, he began to feel better. He rapidly noted a decrease in his sense of impending doom and anxiety. Over the next several days he began to feel significantly more energetic. MM continued to use the invention in the dosing schedule described and his clinical improvement continued over several weeks to the point that he was able to return to work about six weeks later.

Recommended Use: Liposomal Glutathione

1 ounce is 5.56 teaspoons.

1 teaspoon of oral liposomal glutathione reduced contains approximately 440 mg GSH.

Suggested dose depends on body weight. Recommended amounts are for daily use.

Gently stir liposomal glutathione into the liquid of your choice.

Refrigeration after opening is required to prevent deterioration.

Lipoceutical Glutathione-Determine Daily Dose by Body Weight

Under 30 lbs: ¼ teaspoon=110 mg GSH

30-60 lbs: ½ teaspoon=220 mg GSH

60-90 lbs: ¾ teaspoon=330 mg GSH

90-120 lbs: 1 teaspoon=440 mg GSH

120-150 lbs: 1½ teaspoon=660 mg GSH

Over 150 lbs: 2 teaspoons=880 mg GSH

Recommended Use: Liposomal Methylcobalamine

Children: Determine Daily Dose by Body Weight

Replacement formula for children is 75 μg/Kg in the preferred form. Thus a 5 Kg child would receive 375 mcg (μg) of liposomal methylcobalamine. In this situation the dosing syringe would be used to administer 0.48 ml of the methylcobalamine invention.

| Weight in KG | Dose in ml/day | No. of sprays/Day |
|---|---|---|
| 5 | 375 | .75 |
| 10 | 750 | 1.5 |
| 20 | 1500 | 3 |
| 40 | 3000 | 6 |

### REFERENCES

Aguilar, B, Rojas J C, Collados M T, “Metabolism of hip with cardiovascular disease,” Journal Thrombosis Thrombolysis. 2004 October; 18(2):75-87; PMID: 15789174.
Bottiglieri T, S-Adenosyl-L-methionine (SAMe): from the bench to the bedside—molecular basis of a pleiotrophic molecule, American Journal Clinical Nutrition. 2002 November; 76(5):1151S-7S; PMID: 12418493.
Cersosimo R J, Oxaliplatin-associated neuropathy: a review, Annals Pharmacotherapy. 2005 January; 39(1):128-35. Epub 2004 Dec. 8; PMID: 15590869.
Chiang P K, Gordon R K, Tal J, Zeng G C, Doctor B P, Pardhasaradhi K, McCann P P, S-Adenosylmethionine and methylation, FASEB Journal. 1996 March; 10(4):471-8; PMID: 8647346.
Dickinson D A, Forman H J, Glutathione in defense and signaling: lessons from a small thiol, Annals New York Academy Science. 2002 November; 973:488-504; PMID: 12485918
Dröge W, Schulze-Osthoff K, Mihm S, Galter D, Schenk H, Eck H P, Roth S and Gmunder H, Functions of glutathione and glutathione disulfide in immunology and immunopathology. FASEB Journal 1994, 8: 1131-1138.
Droge W, Oxidative stress and aging, Advances Experimental Medicine Biology. 2003; 543:191-200; PMID: 14713123.
Haddad J J, Glutathione depletion is associated with augmenting a proinflammatory signal: evidence for an antioxidant/pro-oxidant mechanism regulating cytokines in the alveolar epithelium, Cytokines Cell Mol Ther. 2000 December; 6(4):177-87; PMID: 11565956.
James S J, Cutler P, Melnyk S, Jernigan S, Janak L, Gaylor D, and Neubrander J Metabolic biomarkers of increased oxidative stress and impaired methylation capacity in children with autism, American Journal Clinical Nutrition 2004; 80:1611-7; PMID: 15585776.
Kamata H, Hirata H, Redox regulation of cellular signaling, Cell Signal. 1999 January; 11(1):1-14; PMID: 10206339.
Lieber C S, Relationships between nutrition, alcohol use, and liver disease, Alcohol Res Health. 2003; 27(3):220-31; PMID: 15535450.
Meganathan R, Ubiquinone biosynthesis in microorganisms, FEMS Microbiology Letters. 2001 Sep. 25; 203(2):131-9; PMID: 11583838.
Meister, A, Glutathione synthesis, In The Enzymes (P. D. Boyer, Ed.), 1974, pp. 671-697. Academic Press, New York.
Mischoulon D and Fava M, Role of S-adenosyl-L-methionine in the treatment of depression: a review of the evidence, American Journal of Clinical Nutrition, 2002 Vol. 76, No. 5, 1158S-1161S.
Peterson J D, Herzenberg L A, Vasquez K, Waltenbaugh C, Glutathione levels in antigen-presenting cells modulate Th1 versus Th2 response patterns, Proceedings National Academy Sci USA. 1998 Mar. 17; 95(6):3071-6; PMID: 9501217.
Selhub J, Homocysteine metabolism, Annual Review Nutrition. 1999; 19:217-46; PMID: 10448523.
Waly M, Olteanu H, Banerjee R, Choi S W, Mason J B, Parker B S, Sukumar S, Shim S, Sharma A, Benzecry J M, Power-Charnitsky V A, Deth R C, Activation of methionine synthase by insulin-like growth factor-1 and dopamine: a target for neurodevelopmental toxins and thimerosal, Molecular Psychiatry. 2004 April; 9(4):358-70; PMID: 14745455.
Warner D S, Sheng H, Batinic-Haberle I, Oxidants, antioxidants and the ischemic brain, Journal Experimental Biology. 2004 August; 207(Pt 18):3221-31; PMID: 15299043.
Watson W P, Munter T, Golding B T, A new role for glutathione: protection of vitamin B12 from depletion by xenobiotics, Chemical Research in Toxicology. 2004 December; 17(12):1562-7; PMID: 15606130.
Witschi A, Reddy S, Stofer B, Lauterburg B H, The systemic availability of oral glutathione, European Journal Clinical Pharmacology. 1992; 43(6):667-9; PMID: 1362956.
Xia L, Cregan A G, Berben L A, Brasch N E, Studies on the formation of glutathionylcobalamin: any free intracellular aquacobalamin is likely to be rapidly and irreversibly converted to glutathionylcobalamin, Inorganic Chemistry. 2004 Oct. 18; 43(21):6848-57; PMID: 15476387.
   Separate Reference Sheet for Abbreviations in Drawings
   Methylation includes in this box in FIGS. 1 and 3 Methylation of DNA, RNA, proteins, membrane phospholipids, neurotransmitters
   FIG. 4 begins with Glutamate
   The abbreviations in the Figures, particularly FIGS. 1 and 3, are as follows:
   THF: tetrahydrofolate
   MS: methionine synthase
   BHMT: betaine-homocysteine methyltransferase
   MAT: methionine adenosyltransferase
   SAM: S-adenosylmethionine
   SAH: S-adenosylhomocysteine
   SAHH: SAH hydrolase
   ADA: adenosine deaminase
   AK: adenosine kinase
   CBS: cystathionine beta synthase
   B12: cyanocobalomine
   meB12: methylcobalamine
   5-CH₃ THF: 5-Methyltetrahydrofolate

What is claimed is:

1. A pharmaceutical composition affecting the production and utilization of neurotransmitters, particularly in the SAM cycle, comprising:
reduced glutathione in a liposomal formulation and methylcobalamine in a liposomal formulation.

2. A pharmaceutical composition affecting the production and utilization of neurotransmitters, particularly in the SAM cycle, comprising:
reduced glutathione in a liposomal formulation and IGF-1 in a liposomal formulation.

3. A pharmaceutical composition affecting the production and utilization of neurotransmitters, particularly in the SAM cycle, comprising:
reduced glutathione in a liposomal formulation, methylcobalamine in a liposomal formulation, and IGF-1 in a liposomal formulation.

4. The composition according to claims 1, 2, 3 or 4, further comprising:
a therapeutic dose of selenium.

5. A method of treatment of neurotransmitter deficiency state in mammalian patients, comprising:
administration of reduced glutathione in a liposomal formulation.

6. A method of treatment of neurotransmitter deficiency state in mammalian patients, comprising:
administration of reduced glutathione in a liposomal formulation and methylcobalamine in a liposomal formulation.

7. A method of treatment of neurotransmitter deficiency state in mammalian patients, comprising:
administration of reduced glutathione in a liposomal formulation and IGF-1 in a liposomal formulation.

8. A method of treatment of neurotransmitter deficiency state in mammalian patients, comprising:
administration of reduced glutathione in a liposomal formulation, administration of methylcobalamine in a liposomal formulation, and IGF-1 in a liposomal formulation.

9. A method of treatment of the clinical conditions accompanying vitamin B-12 deficiency, or homocysteine cycle defects, or cysteine generation defects in mammalian patients, comprising:
administration of reduced glutathione in a liposomal formulation, followed by methylcobalamine in a liposomal formulation.

10. A method of treatment of the clinical conditions accompanying vitamin B-12 deficiency, or homocysteine cycle defects, or cysteine generation defects in mammalian patients, comprising:
administration of reduced glutathione in a liposomal formulation, followed by methylcobalamine in a liposomal formulation and IGF-1 in a liposomal formulation.

11. A method of treatment of autism and related neurodevelopmental disorders in mammalian patients, comprising:
administration of reduced glutathione in a liposomal formulation, followed by methylcobalamine in a liposomal formulation.

12. A method of treatment of autism and related neurodevelopmental disorders in mammalian patients, comprising:
administration of reduced glutathione in a liposomal formulation, followed by methylcobalamine in a liposomal formulation and IGF-1 in a liposomal formulation.

13. The method according to claims 5, 6, 7, 8, 9, 10, 11, or 12 further comprising:
administering a therapeutic dose of Selenium.

14. The method according to claims 5, 6, 7, 8, 9, 10, 11, or 12, further comprising the following step:
assessing the level of at least one neurotransmitter in a mammalian patient.

15. The method according to claims 5, 6, 7, 8, 9, 10, 11, or 12, further comprising the following step:
assessing the level of at least one neurotransmitter in a mammalian patient; and
administering a therapeutic dose of Selenium.
